(19) United States
(12) Patent Application Publication
(10) Pub. No.: US 2023/0304396 A1
(43) Pub. Date: Sep. 28, 2023

(54) METHODS FOR ESTIMATING MAXIMUM RESERVOIR INJECTION PRESSURES, AND RELATED NON-TRANSITORY, COMPUTER-READABLE STORAGE MEDIUMS AND COMPUTER SYSTEMS

(71) Applicant: ExxonMobil Technology and Engineering Company, Spring, TX (US)

(72) Inventors: Kelvin I. AMALOKWU, Houston, TX (US); Brian R. CRAWFORD, The Woodlands, TX (US); Shreerang S. CHHATRE, The Woodlands, TX (US)

(21) Appl. No.: 18/175,763

(22) Filed: Feb. 28, 2023

Related U.S. Application Data
(60) Provisional application No. 63/269,714, filed on Mar. 22, 2022.

(51) Int. Cl. E21B 49/00; G01N 3/08; G01N 19/02; G01N 33/24; E21B 43/16

(52) U.S. Cl. CPC E21B 49/00; G01N 3/08; G01N 19/02; G01N 33/24; E21B 43/16; G01N 2203/0067

(57) ABSTRACT

Systems and methods described herein provide for the estimation of the maximum reservoir pressure at which fluid can be injected into a reservoir before causing conductivity increase due to fracture/fault reactivation. An exemplary method includes computing the maximum reservoir pressure for the location of interest prior to fracture/fault reactivation at a given depleted reservoir pressure based on a computed probability of non-exceedance for a field or laboratory estimate of the maximum reservoir pressure prior to fracture/fault reactivation and a computed pressure distribution including a range of potential maximum reservoir pressures for the location of interest prior to fracture/fault reactivation at the given depleted reservoir pressure. The method also includes outputting the computed maximum reservoir pressure as the estimated maximum reservoir pressure for performing a fluid injection operation for the location of interest.

### CROSS-REFERENCE TO RELATED APPLICATION

This application claims the benefit of U.S. Provisional Application Ser. No. 63/269,714, entitled “METHODS FOR ESTIMATING MAXIMUM RESERVOIR INJECTION PRESSURES, AND RELATED NON-TRANSITORY, COMPUTER-READABLE STORAGE MEDIUMS AND COMPUTER SYSTEMS,” filed Mar. 22, 2022, the disclosure of which is hereby incorporated by reference in its entirety.

### FIELD OF THE INVENTION

The techniques described herein relate generally to the field of hydrocarbon recovery and particularly to the field of enhanced oil recovery (EOR). More specifically, the techniques described herein relate to fluid injection at reservoir pressures that avoid fracture/fault reactivation.

### BACKGROUND OF THE INVENTION

This section is intended to introduce various aspects of the art, which may be associated with embodiments of the present techniques. This discussion is believed to assist in providing a framework to facilitate a better understanding of particular aspects of the present techniques. Accordingly, it should be understood that this section should be read in this light, and not necessarily as admissions of prior art.

The extraction of hydrocarbons from unconventional formations (e.g., tight, oil-bearing formations) is typically achieved using natural reservoir pressure drive (generally referred to as “primary depletion”). However, this approach often results in around 90% of the oil being left behind within the formation. Enhanced oil recovery (EOR) techniques, such as, in particular, fluid injection techniques, present opportunities to significantly improve hydrocarbon recovery. However, during such fluid injection, it is common for new conductive pathways to be created as the pressure within the formation increases. Such conductive pathways then allow the injected fluids to undesirably escape into the formation.

More specifically, such conductive pathways are typically formed in response to the reactivation of existing fractures and/or faults within the formation, which occurs at specific reservoir pressures and is dependent on a complex set of reservoir properties. However, current techniques fail to provide an accurate or efficient means of predicting or estimating the reservoir pressures at which such fracture/fault reactivation will occur. In particular, while microseismic observations provide unequivocal evidence of fracture/fault reactivation due to shear slip induced by stress changes associated with fluid injection, previous techniques have attempted to explain this relationship by invoking a decoupled effect of pressure on the minimum horizontal stress due to lack of pressure diffusion on the injection timescale. However, this approach is inconsistent with linear elastic theory and, therefore, does not provide an accurate means of estimating the maximum reservoir pressure prior to fracture/fault reactivation.

According to current techniques, the maximum reservoir pressure prior to fracture/fault reactivation can be directly estimated in the field by injecting fluid into the formation until there is a sudden loss of reservoir pressure due to permeability enhancement associated with fracture/fault reactivation. However, the EOR process must be already underway to obtain such field estimates. Alternatively, the maximum reservoir pressure prior to fracture/fault reactivation can be estimated ahead of time in the laboratory by using core plugs from the interval of interest to determine the relevant reservoir properties for calculating the reservoir pressure. However, core plugs are not always available for all locations of interest. Moreover, as the degree of heterogeneity within the formation increases, the number of core plugs required to make a reliable estimate correspondingly increases, rendering it even more difficult to rely solely on such lab estimates.

### SUMMARY OF THE INVENTION

An embodiment described herein provides a method for estimating the maximum reservoir pressure at which fluid can be injected into a reservoir before causing conductivity increase due to fracture/fault reactivation, wherein the method is executed via a processor of a computing system, and wherein the method includes: accessing first input data including sonic, density, and mineralogy logs for a location of interest; computing average formation static anisotropic elastic properties for the location of interest based on the first input data; computing a Biot's coefficient and a stress path parameter based on the average formation static anisotropic elastic properties; computing a static anisotropic elastic distribution corresponding to the average formation static anisotropic elastic properties; computing a Biot's coefficient distribution and a stress path distribution based on the static anisotropic elastic distribution; enforcing hysteresis on the stress path distribution; accessing second input data including an overburden stress, a minimum horizontal stress, an initial reservoir pressure, estimated fault/fracture Mohr-Coulomb frictional strength parameters, and a depleted pressure for the location of interest; computing a stress change due to depletion at the location of interest based on the second input data, the computed Biot's coefficient, and the computed stress path parameter; computing a first pressure distribution including a first range of potential maximum reservoir pressures for the location of interest prior to fracture/fault reactivation at the given depleted pressure, wherein the pressure distribution is computed based on the computed stress change due to depletion, the estimated fault/fracture Mohr-Coulomb frictional strength parameters, the computed Biot's coefficient distribution, and the computed stress path distribution with hysteresis enforced; accessing third input data including properties corresponding to a second location for which a field estimate or a laboratory estimate of a maximum reservoir pressure prior to fracture/fault reactivation is available; repeating the computation of the average formation static anisotropic elastic properties, the computation of the Biot's coefficient and the stress path parameter, the computation of the static anisotropic elastic distribution, the computation of the Biot's coefficient distribution and the stress path distribution, the enforcement of the hysteresis on the stress path distribution, and the computation of the stress change due to depletion for the second location; computing a second pressure distribution including a second range of potential maximum reservoir pressures for the second location prior to fracture/fault reactivation at the given depleted pressure; computing a probability of non-exceedance for the field estimate or the laboratory estimate of the first maximum reservoir pressure prior to fracture/fault reactivation for the second location; computing a second maximum reservoir pressure for the location of interest prior to fracture/fault reactivation at the given depleted pressure based on the computed probability of non-exceedance and the computed first pressure distribution; and outputting the computed second maximum reservoir pressure as an estimated maximum reservoir pressure for performing a fluid injection operation for the location of interest. In various embodiments, the method also includes performing the fluid injection operation for the location of interest based on the estimated maximum reservoir pressure that is output by the processor.

Another embodiment described herein provides a computing system including a processor and a non-transitory, computer-readable storage medium. The non-transitory, computer-readable storage medium includes code configured to direct the processor to: access first input data including sonic, density, and mineralogy logs for a location of interest; compute average formation static anisotropic elastic properties for the location of interest based on the first input data; compute a Biot's coefficient and a stress path parameter based on the average formation static anisotropic elastic properties; compute a static anisotropic elastic distribution corresponding to the average formation static anisotropic elastic properties; compute a Biot's coefficient distribution and a stress path distribution based on the static anisotropic elastic distribution; enforce hysteresis on the stress path distribution; access second input data including an overburden stress, a minimum horizontal stress, an initial reservoir pressure, estimated fault/fracture Mohr-Coulomb frictional strength parameters, estimated fault/fracture Mohr-Coulomb frictional strength parameters, and a depleted pressure for the location of interest; compute a stress change due to depletion at the location of interest based on the second input data, the computed Biot's coefficient, and the computed stress path parameter; compute a first pressure distribution including a first range of potential maximum reservoir pressures for the location of interest prior to fracture/fault reactivation at the given depleted pressure, wherein the pressure distribution is computed based on the computed stress change due to depletion, the estimated fault/fracture Mohr-Coulomb frictional strength parameters, the computed Biot's coefficient distribution, and the computed stress path distribution with hysteresis enforced; access third input data including properties corresponding to a second location for which a field estimate or a laboratory estimate of a maximum reservoir pressure prior to fracture/fault reactivation is available; repeat the computation of the average formation static anisotropic elastic properties, the computation of the Biot's coefficient and the stress path parameter, the computation of the static anisotropic elastic distribution, the computation of the Biot's coefficient distribution and the stress path distribution, the enforcement of the hysteresis on the stress path distribution, and the computation of the stress change due to depletion for the second location; compute a second pressure distribution including a second range of potential maximum reservoir pressures for the second location prior to fracture/fault reactivation at the given depleted pressure; compute a probability of non-exceedance for the field estimate or the laboratory estimate of the first maximum reservoir prior to fracture/fault reactivation pressure for the second location; compute a second maximum reservoir pressure for the location of interest prior to fracture/fault reactivation at the given depleted pressure based on the computed probability of non-exceedance and the computed first pressure distribution; and output the computed second maximum reservoir pressure as an estimated maximum reservoir pressure for performing a fluid injection operation for the location of interest. In various embodiments, the code is further configured to direct the processor to perform the fluid injection operation for the location of interest based on the estimated maximum reservoir pressure that is output by the processor.

Another embodiment described herein provides a non-transitory, computer-readable storage medium including program instructions that are executable by a processor to cause the processor to: access first input data including sonic, density, and mineralogy logs for a location of interest; compute average formation static anisotropic elastic properties for the location of interest based on the first input data; compute a Biot's coefficient and a stress path parameter based on the average formation static anisotropic elastic properties; compute a static anisotropic elastic distribution corresponding to the average formation static anisotropic elastic properties; compute a Biot's coefficient distribution and a stress path distribution based on the static anisotropic elastic distribution; enforce hysteresis on the stress path distribution; access second input data including an overburden stress, a minimum horizontal stress, an initial reservoir pressure, estimated fault/fracture Mohr-Coulomb frictional strength parameters, and a depleted pressure for the location of interest; compute a stress change due to depletion at the location of interest based on the second input data, the computed Biot's coefficient, and the computed stress path parameter; compute a first pressure distribution including a first range of potential maximum reservoir pressures for the location of interest prior to fracture/fault reactivation at the given depleted pressure, wherein the pressure distribution is computed based on the computed stress change due to depletion, the estimated fault/fracture Mohr-Coulomb frictional strength parameters, the computed Biot's coefficient distribution, and the computed stress path distribution with hysteresis enforced; access third input data including properties corresponding to a second location for which a field estimate or a laboratory estimate of a maximum reservoir pressure prior to fracture/fault reactivation is available; repeat the computation of the average formation static anisotropic elastic properties, the computation of the Biot's coefficient and the stress path parameter, the computation of the static anisotropic elastic distribution, the computation of the Biot's coefficient distribution and the stress path distribution, the enforcement of the hysteresis on the stress path distribution, and the computation of the stress change due to depletion for the second location; compute a second pressure distribution including a second range of potential maximum reservoir pressures for the second location prior to fracture/fault reactivation at the given depleted pressure; compute a probability of non-exceedance for the field estimate or the laboratory estimate of the first maximum reservoir pressure prior to fracture/fault reactivation for the second location; compute a second maximum reservoir pressure for the location of interest prior to fracture/fault reactivation at the given depleted pressure based on the computed probability of non-exceedance and the computed first pressure distribution; and output the computed second maximum reservoir pressure as an estimated maximum reservoir pressure for performing a fluid injection operation for the location of interest. In various embodiments, the program instructions ae further executable by the processor to cause the processor to perform the fluid injection operation for the location of interest based on the estimated maximum reservoir pressure that is output by the processor.

These and other features and attributes of the disclosed embodiments of the present techniques and their advantageous applications and/or uses will be apparent from the detailed description that follows.

### BRIEF DESCRIPTION OF THE DRAWINGS

To assist those of ordinary skill in the relevant art in making and using the subject matter described herein, reference is made to the appended drawings.

FIG. 1 is a schematic view of an exemplary method for estimating the maximum reservoir pressure at which fluid can be injected into a reservoir to enhance hydrocarbon recovery in accordance with the present techniques;

FIG. 2A shows an exemplary pressure (P**) distribution at a location for which a direct measurement of P** exists;

FIG. 2B shows an exemplary P** distribution at a location of interest for which a direct measurement of P** does not exist;

FIG. 3A is a graph showing an exemplary experimental protocol for measuring the Biot's coefficient, as well as the stress path parameter (and its corresponding hysteresis) for particular formation location according to embodiments described herein;

FIG. 3B is a graph showing an exemplary experimental protocol for measuring the coefficient of friction for a particular formation location according to embodiments described herein;

FIG. 4 is a block diagram of an exemplary cluster computing system that may be utilized to implement the present techniques; and

FIG. 5 is a block diagram of an exemplary non-transitory, computer-readable storage medium that may be used for the storage of data and modules of program instructions for implementing the present techniques.

It should be noted that the figures are merely examples of the present techniques and are not intended to impose limitations on the scope of the present techniques. Further, the figures are generally not drawn to scale, but are drafted for purposes of convenience and clarity in illustrating various aspects of the techniques.

### DETAILED DESCRIPTION OF THE INVENTION

In the following detailed description section, the specific examples of the present techniques are described in connection with preferred embodiments. However, to the extent that the following description is specific to a particular embodiment or a particular use of the present techniques, this is intended to be for exemplary purposes only and simply provides a description of the embodiments. Accordingly, the techniques are not limited to the specific embodiments described below, but rather, include all alternatives, modifications, and equivalents falling within the true spirit and scope of the appended claims.

### Terminology

At the outset, and for ease of reference, certain terms used in this application and their meanings as used in this context are set forth. To the extent a term used herein is not defined below, it should be given the broadest definition those skilled in the art have given that term as reflected in at least one printed publication or issued patent. Further, the present techniques are not limited by the usage of the terms shown below, as all equivalents, synonyms, new developments, and terms or techniques that serve the same or a similar purpose are considered to be within the scope of the present claims.

As used herein, the singular forms “a,” “an,” and “the” mean one or more when applied to any embodiment described herein. The use of “a,” “an,” and/or “the” does not limit the meaning to a single feature unless such a limit is specifically stated.

The term “and/or” placed between a first entity and a second entity means one of (1) the first entity, (2) the second entity, and (3) the first entity and the second entity. Multiple entities listed with “and/or” should be construed in the same manner, i.e., “one or more” of the entities so conjoined. Other entities may optionally be present other than the entities specifically identified by the “and/or” clause, whether related or unrelated to those entities specifically identified. Thus, as a non-limiting example, a reference to “A and/or B,” when used in conjunction with open-ended language such as “including,” may refer, in one embodiment, to A only (optionally including entities other than B); in another embodiment, to B only (optionally including entities other than A); in yet another embodiment, to both A and B (optionally including other entities). These entities may refer to elements, actions, structures, steps, operations, values, and the like.

As used herein, the term “any” means one, some, or all of a specified entity or group of entities, indiscriminately of the quantity.

As used herein, the phrase “based on” does not mean “based only on,” unless expressly specified otherwise. In other words, the phrase “based on” means “based only on,” “based at least on,” and/or “based at least in part on.”

The term “core sample” (or “core plug”) refers to a physical sample collected from a subterranean formation corresponding to a hydrocarbon well. Core samples/plugs can be analyzed to extract core data that represent the geophysical properties of the corresponding subterranean formation. Moreover, various techniques may be used to extract such core data from core samples. Such techniques may include, for example, digitization, resampling, extrapolation, interpolation, curve fitting, and like.

The term “enhanced oil recovery” (EOR) refers to processes for enhancing the recovery of hydrocarbons (e.g., primarily oil) from subterranean reservoirs through the introduction of materials not naturally occurring in the reservoir. Examples of EOR techniques include gas injection, chemical flooding, and thermal recovery. Of particular relevance to the present techniques, gas injection involves injecting gas (e.g., natural gas, nitrogen, and/or carbon dioxide) into a reservoir to increase the flow of oil from the reservoir. Moreover, in some cases, liquid may additionally or alternatively be injected into the formation during such techniques. Therefore, the term “fluid injection” is used herein to refer generally to EOR techniques involving the injection of fluids into a formation.

As used herein, the terms “example,” exemplary,” and “embodiment,” when used with reference to one or more components, features, structures, or methods according to the present techniques, are intended to convey that the described component, feature, structure, or method is an illustrative, non-exclusive example of components, features, structures, or methods according to the present techniques. Thus, the described component, feature, structure, or method is not intended to be limiting, required, or exclusive/exhaustive; and other components, features, structures, or methods, including structurally and/or functionally similar and/or equivalent components, features, structures, or methods, are also within the scope of the present techniques.

As used herein, the term “fluid” refers to gases and liquids, as well as to combinations of gases and liquids, combinations of gases and solids, combinations of liquids and solids, and combinations of gases, liquids, and solids.

The term “formation” refers to a subsurface region including an aggregation of subsurface sedimentary, metamorphic and/or igneous matter, whether consolidated or unconsolidated, and other subsurface matter, whether in a solid, semi-solid, liquid and/or gaseous state, related to the geological development of the subsurface region. A formation can be a body of geologic strata of predominantly one type of rock or a combination of types of rock, or a fraction of strata having substantially common sets of characteristics. A formation can contain one or more hydrocarbon-bearing intervals, generally referred to as “reservoirs.” Note that the terms “formation,” “reservoir,” and “interval” may be used interchangeably, but may generally be used to denote progressively smaller subsurface regions, stages, or volumes. More specifically, a “formation” may generally be the largest subsurface region, while a “reservoir” may generally be a hydrocarbon-bearing stage or interval within the geologic formation that includes a relatively high percentage of oil and gas. Moreover, an “interval” may generally be a sub-region or portion of a reservoir.

The term “fracture” refers to a crack or surface of breakage induced by an applied pressure or stress within a subsurface formation.

The term “gas” is used interchangeably with “vapor,” and is defined as a substance or mixture of substances in the gaseous state as distinguished from the liquid or solid state. Likewise, the term “liquid” means a substance or mixture of substances in the liquid state as distinguished from the gas or solid state.

A “hydrocarbon” is an organic compound that primarily includes the elements hydrogen and carbon, although nitrogen, sulfur, oxygen, metals, or any number of other elements may be present in small amounts. As used herein, the term “hydrocarbon” generally refers to components found in raw natural gas and oil.

The term “overburden stress” refers to the pressure exerted on a formation at a given depth due to the total weight of the rocks and fluids above that depth.

The term “hysteresis” refers to the condition whereby the value of a physical property differs for loading (depletion) and unloading (injection). In cyclic stressing of rock in the laboratory, a hysteresis loop is frequently observed whereby the loading and unloading stress versus strain responses for a given cycle are not coincident, and further, specific hysteresis loops associated with multiple cycles of loading and unloading need not overlay one another.

Generally speaking, the term “pressure” refers to a force acting on a unit area. Pressure is typically provided in units of pounds per square inch (psi).

As used herein, the term “production fluids” refers to fluids removed from a subsurface formation, including hydrocarbon fluids removed from an offshore reservoir.

The term “wellbore” refers to a borehole drilled into a subterranean formation. The borehole may include vertical, deviated, highly deviated, and/or horizontal sections. The term “wellbore” also includes the downhole equipment associated with the borehole, such as the casing strings, production tubing, gas lift valves, and other subsurface equipment. Relatedly, the term “hydrocarbon well” (or simply “well”) includes the wellbore in addition to the wellhead and other associated surface equipment.

As described above, current techniques for estimating the reservoir pressure at which fracture/fault reactivation will occur during fluid injection operations suffer from several limitations. In particular, current techniques for providing field estimates of such reservoir pressure are dependent on the EOR (or other fluid injection) process being already underway, while current techniques for providing lab estimates of such reservoir pressure are dependent on the availability of suitable types of core samples.

Accordingly, the present techniques solve this problem by providing systems and methods to estimate enhanced hydrocarbon recovery fluid injection pressures that avoid reactivation of planes of weakness (e.g., fractures and/or faults) within the formation. In particular, the present techniques provide for the accurate and reliable prediction or estimation of the maximum reservoir pressure at which fluid can be injected into the formation while avoiding the undesirable increase of reservoir conductivity due to fracture/fault reactivation. As described herein, the present techniques enable the estimation of such maximum reservoir pressure in locations where direct field estimates are not available (e.g., because the EOR operation or other fluid injection operation has not yet begun), as well as locations where suitable types of core samples are not available to enable accurate lab estimates. Moreover, according to embodiments described herein, the estimated maximum reservoir pressure is then used to inform the optimal field implementation of the corresponding fluid injection operation, including aiding in the design of the compression/pumping capacity for fluid injection.

As described above, microseismic observations provide unequivocal evidence of fracture/fault reactivation due to shear slip induced by stress changes associated with fluid injection. However, while previous techniques have attempted to explain this relationship by invoking a decoupled effect of pressure on the minimum horizontal stress due to lack of pressure diffusion on the injection timescale, this approach is inconsistent with linear elastic theory and lab measurements, which suggest a mechanical coupling between the pore pressure and the horizontal stresses within the formation. Therefore, according to the present techniques, the fracture/fault reactivation is defined using the concept of hysteresis, while also staying consistent with linear elastic theory, as described further herein.

It should be noted that, although the present techniques are described herein with respect to EOR applications, such techniques are easily extendable to any other application in which fluid is being injected into a formation for hydrocarbon recovery purposes, storage purposes, or any other suitable purpose. As an example, the present techniques may be applied to processes for geologically sequestering carbon dioxide.

### Exemplary Method for Estimating Maximum Reservoir Pressure for EOR Operation

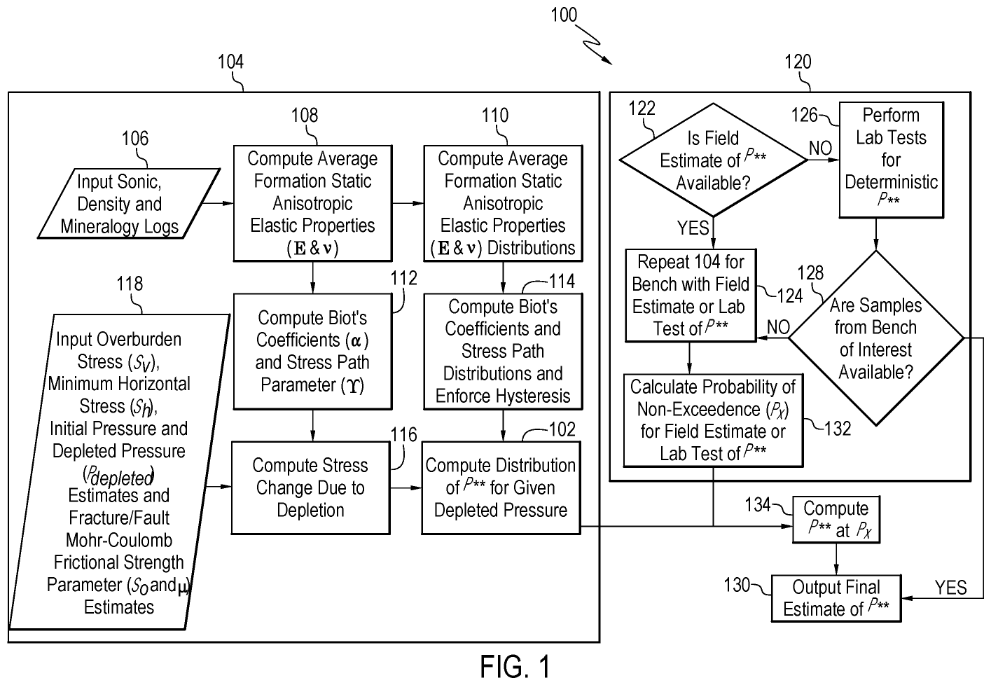

FIG. 1 is a schematic view of an of an exemplary method 100 for estimating the maximum reservoir pressure at which fluid can be injected into a reservoir to enhance hydrocarbon recovery in accordance with the present techniques. In particular, the method 100 enables the estimation of the maximum reservoir pressure prior to fracture/fault reactivation for a location of interest. Such location of interest may include an area of a formation for which neither a direct field estimate nor a lab estimate is available.

The method 100 involves using a coupled analytical approach to calculate a distribution for the pressure at which fracture/fault reactivation (or slipping) occurs in response to fluid injection at a given depleted reservoir pressure, as shown at block 102. Such pressure is denoted as P**, where P** is equal to the depleted pressure (P_{depleted}) plus the change in reservoir pressure (ΔP**) in response to fluid injection. In other words, ΔP** denotes the maximum amount by which the reservoir pressure may be increased during fluid injection without causing optimally-oriented fractures/faults to slip in shear, thus reactivating as a result; and the distribution of P** therefore defines a range of maximum reservoir pressures for efficiently injecting fluid into the reservoir at the given depleted pressure.

According to embodiments described herein, the coupled analytical approach for calculating the distribution of P** at the given depleted pressure accounts for poroelastic coupling between the reservoir pressure and the minimum horizontal stress. In addition, such coupled analytical approach reduces to the non-coupled case when there is no coupling between the reservoir pressure and the minimum horizontal stress. Specifically, assuming a normal faulting regime, and given the overburden stress (S_{V}), the minimum horizontal stress (Sₕ), the depleted reservoir pressure (P_{depleted}), the Biot's coefficient (α), the stress path parameter (γ), the cohesion (Sₒ) and the coefficient of friction (μ) for the location of interest, the change in reservoir pressure (ΔP**) prior to fracture/fault reactivation can be written as shown in Equation (1).

$$Δ {P}^{* *} = \frac{{S}_{V} - {AS}_{h} + α {P}_{depleted} ( A - 1 ) - 2 {S}_{o} \sqrt{A}}{A ( γ - α ) + α} \qquad ( 1 )$$

In Equation (1), the term A is defined by Equation (2).

A=(\sqrt{μ²+1}+μ)² (2)

Turning now to more specific details of the method 100, the first stage 104 of the method 100 involves taking sonic, density, and mineralogy logs, as well as initial estimates of the overburden stress (S_{V}), the minimum horizontal stress (Sₕ), the initial reservoir pressure, and the depleted reservoir pressure (P_{depleted}), as inputs and then calculating the distribution of P** at the given depleted pressure according to Equations (1) and (2), as shown at block 102. In particular, because P** is controlled by a complex combination of subsurface properties that have a range of possible values, a deterministic approach is inadequate to predict P** in the absence of a direct field/lab estimate of P** for the location of interest. Therefore, during the first stage 104 of the method 100, a probabilistic approach is used to capture the possible values of P** and to establish a reasonable range (or distribution) for P**. Moreover, the input values for calculating P** are constrained using, not only the sonic, density, and mineralogy logs and the initial stress and pressure estimates, but also principles of elastic and poroelastic theory and rules of elastic property behavior (e.g., the average range of hysteresis observed in rocks). In addition, stress path parameters are calculated using a Monte Carlo framework, and the coupled analytical solution represented by Equations (1) and (2) then enables the calculation of the P** distribution within the Monte Carlo framework.

More specifically, the first stage 104 of the method 100 involves utilizing the sonic and density logs for the location of interest, which are input at block 106, to compute the average formation static anisotropic elastic properties (E and v) at block 108. At block 110, distributions are then computed for such average formation static anisotropic elastic properties. In addition, the average formation static anisotropic elastic properties from block 108 and the mineralogy logs, which are also input at block 106, are used to compute the Biot's coefficient (α) and the stress path parameter (γ) at block 112. Moreover, the computed Biot's coefficient and the stress path parameter from block 112, as well as the distributions for the average formation static anisotropic elastic properties from block 110, are used to compute distributions for the Biot's coefficient and the stress path parameter at block 114. In addition, during the computation at block 114, hysteresis is enforced to account for the rules of elastic property behavior.

At block 116, the computed Biot's coefficient and stress path parameter from block 112, as well as the initial estimates of the overburden stress, the minimum horizontal stress, the initial reservoir pressure, and the depleted reservoir pressure, which are input at block 118, are used to compute the stress change within the formation due to depletion. Finally, at block 102, the output of block 116 is used, in combination with estimated fault/fracture Mohr-Coulomb frictional strength parameters (Sₒ and μ) (which are also input at block 118) and the computed distributions for the Biot's coefficient and the stress path parameter from block 114, to compute the distribution of P** according to Equations (1) and (2).

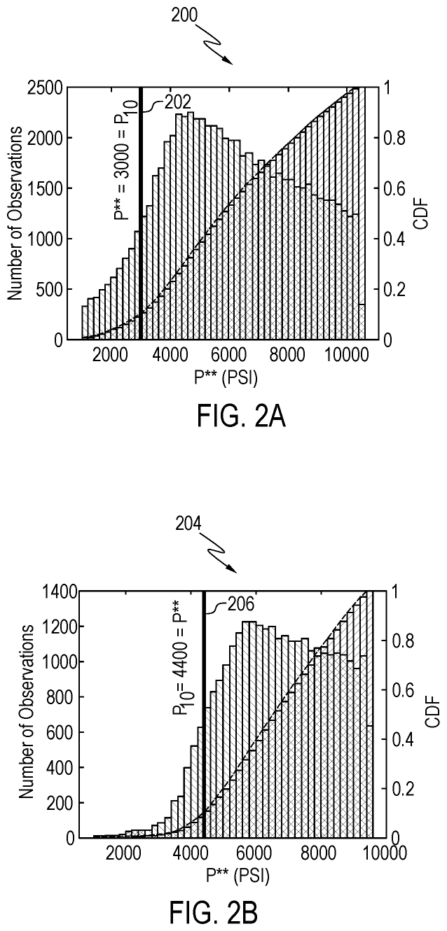

An example of such a distribution of P** is shown in FIGS. 2A and 2B. Specifically, FIG. 2A shows an exemplary P** distribution 200 at a location for which a direct measurement of P** exists (e.g., from field observations or lab testing). For this example, the measured value of P** is 3,000 psi, and the probability of non-exceedance for that P** is equal to P₁₀, as indicated by line 202.

FIG. 2B shows an exemplary P** distribution 204 at a location of interest for which a direct measurement of P** does not exist. Taking the P** distribution 200 from FIG. 2A as the baseline, the P₁₀ within the P** distribution 202 of FIG. 2B is taken as the estimated value of P** at the corresponding location. Therefore, as indicated by line 206, the estimated value of P** is 4,400 psi in this example.

As demonstrated by FIGS. 2A and 2B, once the P** distribution for the location of interest is output from the first stage 104 of the method 100, a narrower range (or single value) of P** is determined using the deterministic approach defined by the second stage 120 of the method 100, as shown in FIG. 1. In particular, the second stage 120 of the method 100 involves first determining whether a direct field estimate of P** exists for the separate location, as shown at block 122. If a direct field estimate of P** does exist for the separate location, the method 100 proceeds to block 124. Otherwise, the method 100 continues at block 126, at which lab tests are performed to compute an estimated value of P** for the separate location. Next, a determination is made at block 128 about whether samples are available from the bench of interest. If samples are available from the bench of interest, the method 100 skips to block 130 with the output of the final estimate of P** for the location of interest. Otherwise, the method 100 proceeds to block 124.

At block 124, the coupled analytic approach defined by the first stage 104 of the method 100 and corresponding Equations (1) and (2) is repeated for the separate location for which the value of P** has been estimated either directly in the field (as determined at block 122) or through the laboratory measurements (as determined at block 126). The probability of non-exceedance (Pₓ) for the field/lab estimate of P** is then computed at block 132 and is provided as the output of the second stage 120 of the method 100. As an example, such computed probability of non-exceedance is P₁₀ for the embodiment represented by FIGS. 2A and 2B.

At block 134, the value of P** for the location of interest is computed using the probability of non-exceedance from block 132. In particular, the P** distribution from block 102 is analyzed to determine the P** value corresponding to the computed probability of non-exceedance from block 132, and such P** value is then output as the final estimated value of P** for the location of interest, as shown at block 130.

With regard to embodiments in which samples are available from the bench of interest, as determined at block 128, specialized lab experiments can be used to estimate the value of P** deterministically. Specifically, measurements on core plugs may be used to make such a deterministic estimation of P**. However, core plugs are not always available for the location of interest. Therefore, the method 100 advantageously provides for the estimation of the value of P** for a location of interest for which no measurements have been taken. Moreover, because it is easier to characterize a relatively homogeneous formation through core plug measurements, in some embodiments, characterization of such a relatively homogeneous reservoir can be used as a basis for estimating the value of P** for a more heterogeneous formation of interest. Additionally or alternatively, lab estimates of the value of P** can be used to validate the output of the method 100.

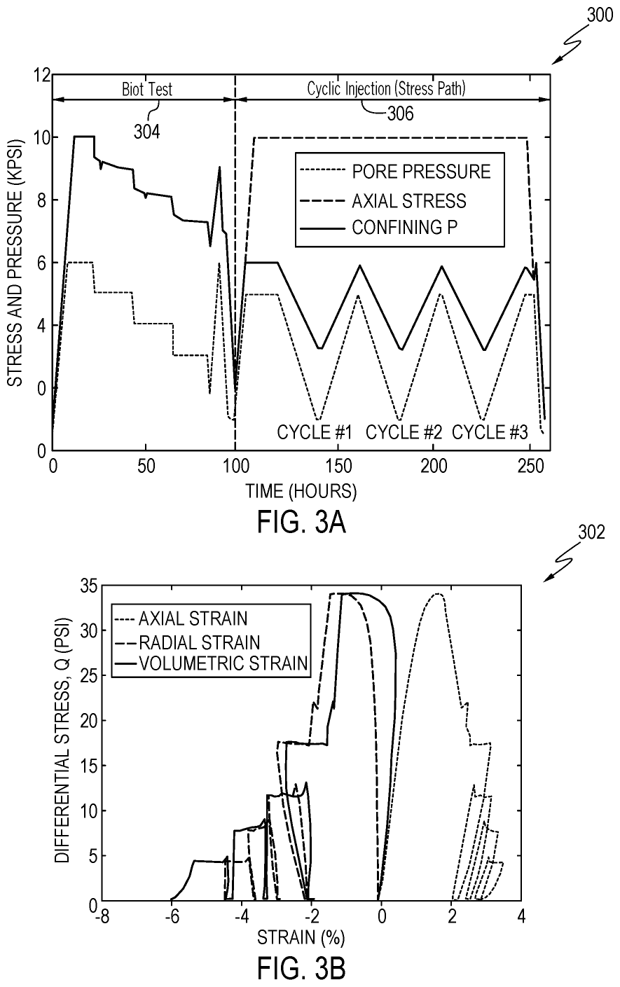

It should be noted that, in order to calculate the value of P** deterministically, direct measurements of the Biot's coefficient, the stress path parameter (and its corresponding hysteresis), and the coefficient of friction are needed, and then the value of P** can be determined using Equations (1) and (2). FIGS. 3A and 3B depict examples of specialized experimental protocols that are used to measure these parameters according to embodiments described herein. Specifically, FIG. 3A is a graph 300 showing an exemplary experimental protocol for measuring the Biot's coefficient, as well as the stress path parameter (and its corresponding hysteresis) for a particular formation location according to embodiments described herein, while FIG. 3B is a graph 302 showing an exemplary experimental protocol for measuring the coefficient of friction for a particular formation location according to embodiments described herein.

As depicted by line 304 in FIG. 3A, the present techniques provide a Biot test in which the Biot's coefficient (α) is measured as the slope of the confining pressure (P_{c}) to the pore pressure (Pₚ) while holding the volumetric strain (εᵥ) constant. In other words, according to the Biot test described herein, the Biot's coefficient is defined by Equation (3).

$${α = \frac{Δ {P}_{c}}{Δ {P}_{p}} \vert}_{{Δ {ε}_{v} = 0}_{}} \qquad ( 3 )$$

Moreover, according to the embodiment represented by FIG. 3A, the Biot's coefficient is equal to 0.88.

As depicted by line 306 in FIG. 3A, the present techniques provide a stress path test in which the stress path parameter is measured as the slope of the confining pressure to the pore pressure under uniaxial strain boundary conditions. This stress path test is performed during both depletion and injection, thus allowing the stress path hysteresis to be measured.

As shown in FIG. 3B, the present techniques also provide a friction coefficient test in which the coefficient of friction is measured by increasing the axial stress on the sample under triaxial loading until the sample undergoes brittle failure. A residual friction measurement protocol is then followed, resulting in the collection of enough data to calculate the coefficient of friction.

Those skilled in the art will appreciate that the exemplary method 100 of FIG. 1 is susceptible to modification without altering the technical effect provided by the present techniques. In practice, the exact manner in which the method is implemented will depend, at least in part, on the details of the specific implementation. For example, in some embodiments, some of the blocks shown in FIG. 1 may be altered or omitted from the method 100 and/or new blocks may be added to the method 100.

### Exemplary Cluster Computing System for Implementing Present Techniques

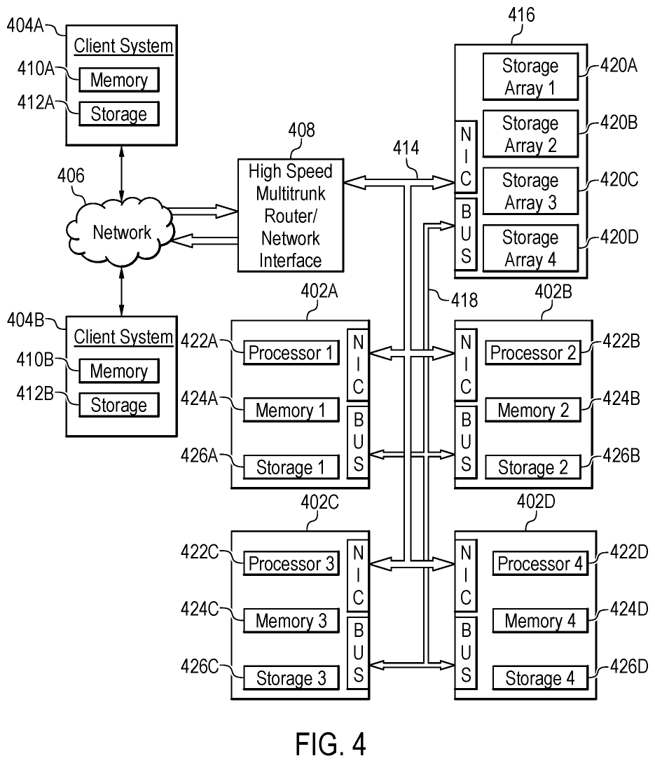

FIG. 4 is a block diagram of an exemplary cluster computing system 400 that may be utilized to implement the present techniques. The exemplary cluster computing system 400 shown in FIG. 4 has four computing units 402A, 402B, 402C, and 402D, each of which may perform calculations for a portion of the present techniques. However, one of ordinary skill in the art will recognize that the cluster computing system 400 is not limited to this configuration, as any number of computing configurations may be selected. For example, a smaller analysis may be run on a single computing unit, such as a workstation, while a large calculation may be run on a cluster computing system 400 having tens, hundreds, thousands, or even more computing units.

The cluster computing system 400 may be accessed from any number of client systems 404A and 404B over a network 406, for example, through a high-speed network interface 408. The computing units 402A to 402D may also function as client systems, providing both local computing support and access to the wider cluster computing system 400.

The network 406 may include a local area network (LAN), a wide area network (WAN), the Internet, or any combinations thereof. Each client system 404A and 404B may include one or more non-transitory, computer-readable storage media for storing the operating code and program instructions that are used to implement the present techniques. For example, each client system 404A and 404B may include a memory device 410A and 410B, which may include random access memory (RAM), read only memory (ROM), and the like. Each client system 404A and 404B may also include a storage device 412A and 412B, which may include any number of hard drives, optical drives, flash drives, or the like.

The high-speed network interface 408 may be coupled to one or more buses in the cluster computing system 400, such as a communications bus 414. The communication bus 414 may be used to communicate instructions and data from the high-speed network interface 408 to a cluster storage system 416 and to each of the computing units 402A to 402D in the cluster computing system 400. The communications bus 414 may also be used for communications among the computing units 402A to 402D and the cluster storage system 416. In addition to the communications bus 414, a high-speed bus 418 can be present to increase the communications rate between the computing units 402A to 402D and/or the cluster storage system 416.

The cluster storage system 416 can have one or more non-transitory, computer-readable storage media, such as storage arrays 420A, 420B, 420C and 420D for the storage of models, data (including core data relating to one or more wells), visual representations, results (such as graphs, charts, and the like used to convey results obtained using the present techniques), code, and other information concerning the implementation of the present techniques. The storage arrays 420A to 420D may include any combinations of hard drives, optical drives, flash drives, or the like.

Each computing unit 402A to 402D can have a processor 422A, 422B, 422C and 422D and associated local non-transitory, computer-readable storage media, such as a memory device 424A, 424B, 424C and 424D and a storage device 426A, 426B, 426C and 426D. Each processor 422A to 422D may be a multiple core unit, such as a multiple core central processing unit (CPU) or a graphics processing unit (GPU). Each memory device 424A to 424D may include ROM and/or RAM used to store program instructions for directing the corresponding processor 422A to 422D to implement the present techniques. Each storage device 426A to 426D may include one or more hard drives, optical drives, flash drives, or the like. In addition, each storage device 426A to 426D may be used to provide storage for models, intermediate results, data, images, or code associated with operations, including code used to implement the present techniques.

The present techniques are not limited to the architecture or unit configuration illustrated in FIG. 4. For example, any suitable processor-based device may be utilized for implementing all or a portion of embodiments of the present techniques, including without limitation personal computers, laptop computers, computer workstations, mobile devices, and multi-processor servers or workstations with (or without) shared memory. Moreover, embodiments may be implemented on application specific integrated circuits (ASICs) or very-large-scale integrated (VLSI) circuits. In fact, persons of ordinary skill in the art may utilize any number of suitable structures capable of executing logical operations according to embodiments described herein.

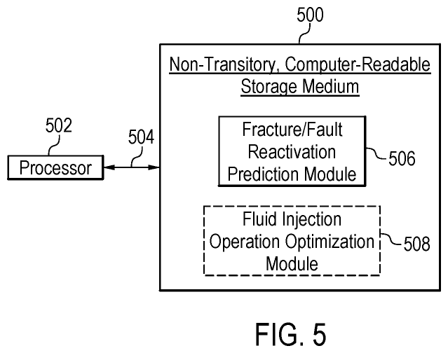

FIG. 5 is a block diagram of an exemplary non-transitory, computer-readable storage medium 500 that may be used for the storage of data and modules of program instructions for implementing the present techniques. The non-transitory, computer-readable storage medium 500 may include a memory device, a hard disk, and/or any number of other devices, as described herein. A processor 502 may access the non-transitory, computer-readable storage medium 500 over a bus or network 504. While the non-transitory, computer-readable storage medium 500 may include any number of modules (and sub-modules) for implementing the present techniques, in some embodiments, the non-transitory, computer-readable storage medium 500 includes a fracture/fault reactivation prediction module 506 for estimating a maximum reservoir pressure at which fluid injection may be performed at a given depleted pressure for a location of interest prior to the undesirable reactivation of fractures and/or faults within the formation. More specifically, the fracture/fault reactivation prediction module 506 may direct the processor 502 to perform the following: (1) access first input data including sonic, density, and mineralogy logs for the location of interest; (2) compute average formation static anisotropic elastic properties for the location of interest based on the first input data; (3) compute the Biot's coefficient and the stress path parameter based on the average formation static anisotropic elastic properties; (4) compute a static anisotropic elastic distribution corresponding to the average formation static anisotropic elastic properties; (5) compute a Biot's coefficient distribution and a stress path distribution based on the static anisotropic elastic distribution; (6) enforce hysteresis on the stress path distribution; (7) access second input data including the overburden stress, the minimum horizontal stress, the initial reservoir pressure, estimated fault/fracture Mohr-Coulomb frictional strength parameters, and the depleted pressure for the location of interest; (8) compute the stress change due to depletion at the location of interest based on the second input data, the computed Biot's coefficient, and the computed stress path parameter; (9) compute a first pressure distribution including a first range of potential maximum reservoir pressures for the location of interest prior to fracture/fault reactivation at the given depleted pressure, where the pressure distribution is computed based on the computed stress change due to depletion, the estimated fault/fracture Mohr-Coulomb frictional strength parameters, the computed Biot's coefficient distribution, and the computed stress path distribution with hysteresis enforced; (10) access third input data including properties corresponding to a second location for which a field estimate or a laboratory estimate of the maximum reservoir pressure prior to fracture/fault reactivation is available; (11) repeat the computation of the average formation static anisotropic elastic properties, the computation of the Biot's coefficient and the stress path parameter, the computation of the static anisotropic elastic distribution, the computation of the Biot's coefficient distribution and the stress path distribution, the enforcement of the hysteresis on the stress path distribution, and the computation of the stress change due to depletion for the second location; (12) compute a second pressure distribution including a second range of potential maximum reservoir pressures for the second location prior to fracture/fault reactivation at the given depleted pressure; (13) compute a probability of non-exceedance for the field estimate or the laboratory estimate of the first maximum reservoir pressure for the second location; (14) compute a second maximum reservoir pressure for the location of interest prior to fracture/fault reactivation at the given depleted pressure based on the computed probability of non-exceedance and the computed first pressure distribution for the location of interest; and (15) output the computed second maximum reservoir pressure as an estimated maximum reservoir pressure for efficiently performing a fluid injection operation for the location of interest. In addition, in some embodiments, the fracture/fault reactivation prediction module 506 may direct the processor 502 to compute a laboratory estimate for the second maximum reservoir pressure for the location of interest using core samples from the location of interest and to utilize the laboratory estimate to validate the estimated maximum reservoir pressure that is output by the processor 502. Moreover, in some embodiments, the fracture/fault reactivation prediction module 506 may direct the processor 502 to compute the field estimate or the laboratory estimate of the first maximum reservoir pressure for the second location. This may include, for example, performing the Biot test, the stress path test, and/or the friction coefficient test described herein.

Furthermore, in some embodiments, the non-transitory, computer-readable storage medium 500 includes a fluid injection operation optimization module 506 for utilizing the output of the fracture/fault reactivation prediction module 506 to design or finetune the parameters for performing the fluid injection operation. This may include, for example, designing the compression/pumping capacity for the fluid injection operation. In addition, in some embodiments, the fluid injection operation optimization module 506 also directs the processor 502 to perform (or direct the performance of) the fluid injection operation for the location of interest based on the output of the fracture/fault reactivation prediction module 506. In this manner, the techniques described herein provide a practical application that directly improves the fluid injection process for the particular location of interest, enabling fluid to be injected at injection pressures that avoid the reactivation of planes of weakness (e.g., fractures and/or faults) within the formation.

### Embodiments of Present Techniques

In one or more embodiments, the present techniques may be susceptible to various modifications and alternative forms, such as the following embodiments as noted in paragraphs 1 to 20.

1. A method for estimating the maximum reservoir pressure at which fluid can be injected into a reservoir before causing conductivity increase due to fracture/fault reactivation, where the method is executed via a processor of a computing system, and where the method includes: accessing first input data including sonic, density, and mineralogy logs for a location of interest; computing average formation static anisotropic elastic properties for the location of interest based on the first input data; computing a Biot's coefficient and a stress path parameter based on the average formation static anisotropic elastic properties; computing a static anisotropic elastic distribution corresponding to the average formation static anisotropic elastic properties; computing a Biot's coefficient distribution and a stress path distribution based on the static anisotropic elastic distribution; enforcing hysteresis on the stress path distribution; accessing second input data including an overburden stress, a minimum horizontal stress, an initial reservoir pressure, estimated fault/fracture Mohr-Coulomb frictional strength parameters, and a depleted pressure for the location of interest; computing a stress change due to depletion at the location of interest based on the second input data, the computed Biot's coefficient, and the computed stress path parameter; computing a first pressure distribution including a first range of potential maximum reservoir pressures for the location of interest prior to fracture/fault reactivation at the given depleted pressure, where the pressure distribution is computed based on the computed stress change due to depletion, the estimated fault/fracture Mohr-Coulomb frictional strength parameters, the computed Biot's coefficient distribution, and the computed stress path distribution with hysteresis enforced; accessing third input data including properties corresponding to a second location for which a field estimate or a laboratory estimate of a maximum reservoir pressure prior to fracture/fault reactivation is available; repeating the computation of the average formation static anisotropic elastic properties, the computation of the Biot's coefficient and the stress path parameter, the computation of the static anisotropic elastic distribution, the computation of the Biot's coefficient distribution and the stress path distribution, the enforcement of the hysteresis on the stress path distribution, and the computation of the stress change due to depletion for the second location; computing a second pressure distribution including a second range of potential maximum reservoir pressures for the second location prior to fracture/fault reactivation at the given depleted pressure; computing a probability of non-exceedance for the field estimate or the laboratory estimate of the first maximum reservoir pressure prior to fracture/fault reactivation for the second location; computing a second maximum reservoir pressure for the location of interest prior to fracture/fault reactivation at the given depleted pressure based on the computed probability of non-exceedance and the computed first pressure distribution; and outputting the computed second maximum reservoir pressure as an estimated maximum reservoir pressure for performing a fluid injection operation for the location of interest.

2. The method of paragraph 1, further including performing the fluid injection operation for the location of interest based on the estimated maximum reservoir pressure that is output via the method.

3. The method of paragraph 2, where performing the fluid injection operation based on the computed second maximum reservoir pressure includes designing a compression/pumping capacity for the fluid injection operation based on the computed second maximum reservoir pressure.

4. The method of any of paragraphs 1 to 3, including computing the stress path parameter using a Monte Carlo framework.

5. The method of any of paragraphs 1 to 4, including generating the third input data by computing the field estimate or the laboratory estimate of the first maximum reservoir pressure for the second location.

6. The method of paragraph 5, where computing the field estimate or the laboratory estimate of the first maximum reservoir pressure includes performing a Biot test to determine the Biot's coefficient for the second location, and where performing the Biot test includes measuring the Biot's coefficient as a slope of a confining pressure to a pore pressure, while holding volumetric strain constant.

7. The method of paragraph 5, where computing the field estimate or the laboratory estimate of the first maximum reservoir pressure further includes performing a stress path test to determine a stress path parameter for the second location, where performing the stress path test includes measuring the stress path parameter as a slope of a confining pressure to a pore pressure under uniaxial strain boundary conditions, and where the stress path test is performed during both depletion and injection to allow the hysteresis to be measured.

8. The method of paragraph 5, where computing the field estimate or the laboratory estimate of the first maximum reservoir pressure further includes performing a friction coefficient test to determine the coefficient of friction for the second location, and where performing the friction coefficient test includes measuring the coefficient of friction by: increasing an axial stress on a sample under triaxial loading until the sample undergoes brittle failure; implementing a residual friction measurement protocol to collect friction data; and computing the coefficient of friction based on the collected friction data.

9. The method of any of paragraphs 1 to 8, including: computing a laboratory estimate for the second maximum reservoir pressure for the location of interest using core samples from the location of interest; and utilizing the laboratory estimate to validate the estimated maximum reservoir pressure that is output via the method.

10. A computing system, including: a processor; and a non-transitory, computer-readable storage medium, including code configured to direct the processor to: access first input data including sonic, density, and mineralogy logs for a location of interest; compute average formation static anisotropic elastic properties for the location of interest based on the first input data; compute a Biot's coefficient and a stress path parameter based on the average formation static anisotropic elastic properties; compute a static anisotropic elastic distribution corresponding to the average formation static anisotropic elastic properties; compute a Biot's coefficient distribution and a stress path distribution based on the static anisotropic elastic distribution; enforce hysteresis on the stress path distribution; access second input data including an overburden stress, a minimum horizontal stress, an initial reservoir pressure, estimated fault/fracture Mohr-Coulomb frictional strength parameters, estimated fault/fracture Mohr-Coulomb frictional strength parameters, and a depleted pressure for the location of interest; compute a stress change due to depletion at the location of interest based on the second input data, the computed Biot's coefficient, and the computed stress path parameter; compute a first pressure distribution including a first range of potential maximum reservoir pressures for the location of interest prior to fracture/fault reactivation at the given depleted pressure, where the pressure distribution is computed based on the computed stress change due to depletion, the estimated fault/fracture Mohr-Coulomb frictional strength parameters, the computed Biot's coefficient distribution, and the computed stress path distribution with hysteresis enforced; access third input data including properties corresponding to a second location for which a field estimate or a laboratory estimate of a maximum reservoir pressure prior to fracture/fault reactivation is available; repeat the computation of the average formation static anisotropic elastic properties, the computation of the Biot's coefficient and the stress path parameter, the computation of the static anisotropic elastic distribution, the computation of the Biot's coefficient distribution and the stress path distribution, the enforcement of the hysteresis on the stress path distribution, and the computation of the stress change due to depletion for the second location; compute a second pressure distribution including a second range of potential maximum reservoir pressures for the second location prior to fracture/fault reactivation at the given depleted pressure; compute a probability of non-exceedance for the field estimate or the laboratory estimate of the first maximum reservoir prior to fracture/fault reactivation pressure for the second location; compute a second maximum reservoir pressure for the location of interest prior to fracture/fault reactivation at the given depleted pressure based on the computed probability of non-exceedance and the computed first pressure distribution; and output the computed second maximum reservoir pressure as an estimated maximum reservoir pressure for performing a fluid injection operation for the location of interest.

11. The computing system of paragraph 10, where the non-transitory, computer-readable storage medium includes code configured to direct the processor to perform the fluid injection operation for the location of interest based on the estimated maximum reservoir pressure that is output by the processor.

12. The computing system of paragraph 10 or 11, where the non-transitory, computer-readable storage medium includes code configured to direct the processor to compute the stress path parameter using a Monte Carlo framework.

13. The computing system of any of paragraphs 10 to 12, where the non-transitory, computer-readable storage medium includes code configured to direct the processor to generate the third input data by computing the field estimate or the laboratory estimate of the first maximum reservoir pressure for the second location.

14. The computing system of paragraph 13, where the non-transitory, computer-readable storage medium includes code configured to direct the processor to compute the field estimate or the laboratory estimate of the first maximum reservoir pressure for the second location by performing a Biot test to determine the Biot's coefficient for the second location, where performing the Biot test includes measuring the Biot's coefficient as a slope of a confining pressure to a pore pressure, while holding volumetric strain constant.

15. The computing system of any of paragraphs 10 to 14, where the non-transitory, computer-readable storage medium includes code configured to direct the processor to: compute a laboratory estimate for the second maximum reservoir pressure for the location of interest using core samples from the location of interest; and utilize the laboratory estimate to validate the estimated maximum reservoir pressure that is output by the processor.

16. A non-transitory, computer-readable storage medium, including program instructions that are executable by a processor to cause the processor to: access first input data including sonic, density, and mineralogy logs for a location of interest; compute average formation static anisotropic elastic properties for the location of interest based on the first input data; compute a Biot's coefficient and a stress path parameter based on the average formation static anisotropic elastic properties; compute a static anisotropic elastic distribution corresponding to the average formation static anisotropic elastic properties; compute a Biot's coefficient distribution and a stress path distribution based on the static anisotropic elastic distribution; enforce hysteresis on the stress path distribution; access second input data including an overburden stress, a minimum horizontal stress, an initial reservoir pressure, estimated fault/fracture Mohr-Coulomb frictional strength parameters, and a depleted pressure for the location of interest; compute a stress change due to depletion at the location of interest based on the second input data, the computed Biot's coefficient, and the computed stress path parameter; compute a first pressure distribution including a first range of potential maximum reservoir pressures for the location of interest prior to fracture/fault reactivation at the given depleted pressure, where the pressure distribution is computed based on the computed stress change due to depletion, the estimated fault/fracture Mohr-Coulomb frictional strength parameters, the computed Biot's coefficient distribution, and the computed stress path distribution with hysteresis enforced; access third input data including properties corresponding to a second location for which a field estimate or a laboratory estimate of a maximum reservoir pressure prior to fracture/fault reactivation is available; repeat the computation of the average formation static anisotropic elastic properties, the computation of the Biot's coefficient and the stress path parameter, the computation of the static anisotropic elastic distribution, the computation of the Biot's coefficient distribution and the stress path distribution, the enforcement of the hysteresis on the stress path distribution, and the computation of the stress change due to depletion for the second location; compute a second pressure distribution including a second range of potential maximum reservoir pressures for the second location prior to fracture/fault reactivation at the given depleted pressure; compute a probability of non-exceedance for the field estimate or the laboratory estimate of the first maximum reservoir pressure prior to fracture/fault reactivation for the second location; compute a second maximum reservoir pressure for the location of interest prior to fracture/fault reactivation at the given depleted pressure based on the computed probability of non-exceedance and the computed first pressure distribution; and output the computed second maximum reservoir pressure as an estimated maximum reservoir pressure for performing a fluid injection operation for the location of interest.

17. The non-transitory, computer-readable storage medium of paragraph 16, where the non-transitory, computer-readable storage medium includes code configured to direct the processor to perform the fluid injection operation for the location of interest based on the estimated maximum reservoir pressure that is output by the processor.

18. The non-transitory, computer-readable storage medium of paragraph 16 or 17, where the non-transitory, computer-readable storage medium includes code configured to direct the processor to generate the third input data by computing the field estimate or the laboratory estimate of the first maximum reservoir pressure for the second location.

19. The non-transitory, computer-readable storage medium of paragraph 18, where the non-transitory, computer-readable storage medium includes code configured to direct the processor to compute the field estimate or the laboratory estimate of the first maximum reservoir pressure for the second location by performing a Biot test to determine the Biot's coefficient for the second location, where performing the Biot test includes measuring the Biot's coefficient as a slope of a confining pressure to a pore pressure, while holding volumetric strain constant.

20. The non-transitory, computer-readable storage medium of any of paragraphs 16 to 19, where the non-transitory, computer-readable storage medium includes code configured to direct the processor to: compute a laboratory estimate for the second maximum reservoir pressure for the location of interest using core samples from the location of interest; and utilize the laboratory estimate to validate the estimated maximum reservoir pressure that is output by the processor.

While the embodiments described herein are well-calculated to achieve the advantages set forth, it will be appreciated that such embodiments are susceptible to modification, variation, and change without departing from the spirit thereof In other words, the particular embodiments described herein are illustrative only, as the teachings of the present techniques may be modified and practiced in different but equivalent manners apparent to those skilled in the art having the benefit of the teachings herein. Moreover, the systems and methods illustratively disclosed herein may suitably be practiced in the absence of any element that is not specifically disclosed herein and/or any optional element disclosed herein. While compositions and methods are described in terms of “comprising” or “including” various components or steps, the compositions and methods can also “consist essentially of” or “consist of” the various components and steps. Indeed, the present techniques include all alternatives, modifications, and equivalents falling within the true spirit and scope of the appended claims.

What is claimed is:

1. A method for estimating the maximum reservoir pressure at which fluid can be injected into a reservoir before causing conductivity increase due to fracture/fault reactivation, wherein the method is executed via a processor of a computing system, and wherein the method comprises:
accessing first input data comprising sonic, density, and mineralogy logs for a location of interest;
computing average formation static anisotropic elastic properties for the location of interest based on the first input data;
computing a Biot's coefficient and a stress path parameter based on the average formation static anisotropic elastic properties;
computing a static anisotropic elastic distribution corresponding to the average formation static anisotropic elastic properties;
computing a Biot's coefficient distribution and a stress path distribution based on the static anisotropic elastic distribution;
enforcing hysteresis on the stress path distribution;
accessing second input data comprising an overburden stress, a minimum horizontal stress, an initial reservoir pressure, estimated fault/fracture Mohr-Coulomb frictional strength parameters, and a depleted pressure for the location of interest;
computing a stress change due to depletion at the location of interest based on the second input data, the computed Biot's coefficient, and the computed stress path parameter;
computing a first pressure distribution comprising a first range of potential maximum reservoir pressures for the location of interest prior to fracture/fault reactivation at the given depleted pressure, wherein the pressure distribution is computed based on the computed stress change due to depletion, the estimated fault/fracture Mohr-Coulomb frictional strength parameters, the computed Biot's coefficient distribution, and the computed stress path distribution with hysteresis enforced;
accessing third input data comprising properties corresponding to a second location for which a field estimate or a laboratory estimate of a maximum reservoir pressure prior to fracture/fault reactivation is available;
repeating the computation of the average formation static anisotropic elastic properties, the computation of the Biot's coefficient and the stress path parameter, the computation of the static anisotropic elastic distribution, the computation of the Biot's coefficient distribution and the stress path distribution, the enforcement of the hysteresis on the stress path distribution, and the computation of the stress change due to depletion for the second location;
computing a second pressure distribution comprising a second range of potential maximum reservoir pressures for the second location prior to fracture/fault reactivation at the given depleted pressure;
computing a probability of non-exceedance for the field estimate or the laboratory estimate of the first maximum reservoir pressure prior to fracture/fault reactivation for the second location;
computing a second maximum reservoir pressure for the location of interest prior to fracture/fault reactivation at the given depleted pressure based on the computed probability of non-exceedance and the computed first pressure distribution; and
outputting the computed second maximum reservoir pressure as an estimated maximum reservoir pressure for performing a fluid injection operation for the location of interest.

2. The method of claim 1, further comprising performing the fluid injection operation for the location of interest based on the estimated maximum reservoir pressure that is output via the method.

3. The method of claim 2, wherein performing the fluid injection operation based on the computed second maximum reservoir pressure comprises designing a compression/pumping capacity for the fluid injection operation based on the computed second maximum reservoir pressure.

4. The method of claim 1, comprising computing the stress path parameter using a Monte Carlo framework.

5. The method of claim 1, comprising generating the third input data by computing the field estimate or the laboratory estimate of the first maximum reservoir pressure for the second location.

6. The method of claim 5, wherein computing the field estimate or the laboratory estimate of the first maximum reservoir pressure comprises performing a Biot test to determine the Biot's coefficient for the second location, and wherein performing the Biot test comprises measuring the Biot's coefficient as a slope of a confining pressure to a pore pressure, while holding volumetric strain constant.

7. The method of claim 5, wherein computing the field estimate or the laboratory estimate of the first maximum reservoir pressure further comprises performing a stress path test to determine a stress path parameter for the second location, wherein performing the stress path test comprises measuring the stress path parameter as a slope of a confining pressure to a pore pressure under uniaxial strain boundary conditions, and wherein the stress path test is performed during both depletion and injection to allow the hysteresis to be measured.

8. The method of claim 5, wherein computing the field estimate or the laboratory estimate of the first maximum reservoir pressure further comprises performing a friction coefficient test to determine the coefficient of friction for the second location, and wherein performing the friction coefficient test comprises measuring the coefficient of friction by:
increasing an axial stress on a sample under triaxial loading until the sample undergoes brittle failure;
implementing a residual friction measurement protocol to collect friction data; and
computing the coefficient of friction based on the collected friction data.

9. The method of claim 1, comprising:
computing a laboratory estimate for the second maximum reservoir pressure for the location of interest using core samples from the location of interest; and
utilizing the laboratory estimate to validate the estimated maximum reservoir pressure that is output via the method.

10. A computing system, comprising:
a processor; and
a non-transitory, computer-readable storage medium, comprising code configured to direct the processor to:
access first input data comprising sonic, density, and mineralogy logs for a location of interest;
compute average formation static anisotropic elastic properties for the location of interest based on the first input data;
compute a Biot's coefficient and a stress path parameter based on the average formation static anisotropic elastic properties;
compute a static anisotropic elastic distribution corresponding to the average formation static anisotropic elastic properties;
compute a Biot's coefficient distribution and a stress path distribution based on the static anisotropic elastic distribution;
enforce hysteresis on the stress path distribution;
access second input data comprising an overburden stress, a minimum horizontal stress, an initial reservoir pressure, estimated fault/fracture Mohr-Coulomb frictional strength parameters, estimated fault/fracture Mohr-Coulomb frictional strength parameters, and a depleted pressure for the location of interest;
compute a stress change due to depletion at the location of interest based on the second input data, the computed Biot's coefficient, and the computed stress path parameter;
compute a first pressure distribution comprising a first range of potential maximum reservoir pressures for the location of interest prior to fracture/fault reactivation at the given depleted pressure, wherein the pressure distribution is computed based on the computed stress change due to depletion, the estimated fault/fracture Mohr-Coulomb frictional strength parameters, the computed Biot's coefficient distribution, and the computed stress path distribution with hysteresis enforced;
access third input data comprising properties corresponding to a second location for which a field estimate or a laboratory estimate of a maximum reservoir pressure prior to fracture/fault reactivation is available;
repeat the computation of the average formation static anisotropic elastic properties, the computation of the Biot's coefficient and the stress path parameter, the computation of the static anisotropic elastic distribution, the computation of the Biot's coefficient distribution and the stress path distribution, the enforcement of the hysteresis on the stress path distribution, and the computation of the stress change due to depletion for the second location;
compute a second pressure distribution comprising a second range of potential maximum reservoir pressures for the second location prior to fracture/fault reactivation at the given depleted pressure;
compute a probability of non-exceedance for the field estimate or the laboratory estimate of the first maximum reservoir prior to fracture/fault reactivation pressure for the second location;
compute a second maximum reservoir pressure for the location of interest prior to fracture/fault reactivation at the given depleted pressure based on the computed probability of non-exceedance and the computed first pressure distribution; and
output the computed second maximum reservoir pressure as an estimated maximum reservoir pressure for performing a fluid injection operation for the location of interest.

11. The computing system of claim 10, wherein the non-transitory, computer-readable storage medium comprises code configured to direct the processor to perform the fluid injection operation for the location of interest based on the estimated maximum reservoir pressure that is output by the processor.

12. The computing system of claim 10, wherein the non-transitory, computer-readable storage medium comprises code configured to direct the processor to compute the stress path parameter using a Monte Carlo framework.

13. The computing system of claim 10, wherein the non-transitory, computer-readable storage medium comprises code configured to direct the processor to generate the third input data by computing the field estimate or the laboratory estimate of the first maximum reservoir pressure for the second location.

14. The computing system of claim 13, wherein the non-transitory, computer-readable storage medium comprises code configured to direct the processor to compute the field estimate or the laboratory estimate of the first maximum reservoir pressure for the second location by performing a Biot test to determine the Biot' s coefficient for the second location, wherein performing the Biot test comprises measuring the Biot's coefficient as a slope of a confining pressure to a pore pressure, while holding volumetric strain constant.

15. The computing system of claim 10, wherein the non-transitory, computer-readable storage medium comprises code configured to direct the processor to:
compute a laboratory estimate for the second maximum reservoir pressure for the location of interest using core samples from the location of interest; and
utilize the laboratory estimate to validate the estimated maximum reservoir pressure that is output by the processor.

16. A non-transitory, computer-readable storage medium, comprising program instructions that are executable by a processor to cause the processor to:
access first input data comprising sonic, density, and mineralogy logs for a location of interest;
compute average formation static anisotropic elastic properties for the location of interest based on the first input data;
compute a Biot's coefficient and a stress path parameter based on the average formation static anisotropic elastic properties;
compute a static anisotropic elastic distribution corresponding to the average formation static anisotropic elastic properties;
compute a Biot's coefficient distribution and a stress path distribution based on the static anisotropic elastic distribution;
enforce hysteresis on the stress path distribution;
access second input data comprising an overburden stress, a minimum horizontal stress, an initial reservoir pressure, estimated fault/fracture Mohr-Coulomb frictional strength parameters, and a depleted pressure for the location of interest;
compute a stress change due to depletion at the location of interest based on the second input data, the computed Biot's coefficient, and the computed stress path parameter;
compute a first pressure distribution comprising a first range of potential maximum reservoir pressures for the location of interest prior to fracture/fault reactivation at the given depleted pressure, wherein the pressure distribution is computed based on the computed stress change due to depletion, the estimated fault/fracture Mohr-Coulomb frictional strength parameters, the computed Biot's coefficient distribution, and the computed stress path distribution with hysteresis enforced;
access third input data comprising properties corresponding to a second location for which a field estimate or a laboratory estimate of a maximum reservoir pressure prior to fracture/fault reactivation is available;
repeat the computation of the average formation static anisotropic elastic properties, the computation of the Biot's coefficient and the stress path parameter, the computation of the static anisotropic elastic distribution, the computation of the Biot's coefficient distribution and the stress path distribution, the enforcement of the hysteresis on the stress path distribution, and the computation of the stress change due to depletion for the second location;
compute a second pressure distribution comprising a second range of potential maximum reservoir pressures for the second location prior to fracture/fault reactivation at the given depleted pressure;
compute a probability of non-exceedance for the field estimate or the laboratory estimate of the first maximum reservoir pressure prior to fracture/fault reactivation for the second location;
compute a second maximum reservoir pressure for the location of interest prior to fracture/fault reactivation at the given depleted pressure based on the computed probability of non-exceedance and the computed first pressure distribution; and
output the computed second maximum reservoir pressure as an estimated maximum reservoir pressure for performing a fluid injection operation for the location of interest.

17. The non-transitory, computer-readable storage medium of claim 16, wherein the non-transitory, computer-readable storage medium comprises code configured to direct the processor to perform the fluid injection operation for the location of interest based on the estimated maximum reservoir pressure that is output by the processor.

18. The non-transitory, computer-readable storage medium of claim 16, wherein the non-transitory, computer-readable storage medium comprises code configured to direct the processor to generate the third input data by computing the field estimate or the laboratory estimate of the first maximum reservoir pressure for the second location.

19. The non-transitory, computer-readable storage medium of claim 18, wherein the non-transitory, computer-readable storage medium comprises code configured to direct the processor to compute the field estimate or the laboratory estimate of the first maximum reservoir pressure for the second location by performing a Biot test to determine the Biot's coefficient for the second location, wherein performing the Biot test comprises measuring the Biot's coefficient as a slope of a confining pressure to a pore pressure, while holding volumetric strain constant.

20. The non-transitory, computer-readable storage medium of claim 16, wherein the non-transitory, computer-readable storage medium comprises code configured to direct the processor to:
compute a laboratory estimate for the second maximum reservoir pressure for the location of interest using core samples from the location of interest; and
utilize the laboratory estimate to validate the estimated maximum reservoir pressure that is output by the processor.
